(19) United States
(12) Patent Application Publication
(10) Pub. No.: US 2019/0000766 A1
(43) Pub. Date: Jan. 3, 2019

(54) ABUSE DETERRENT SOFT CHEWABLE DRUG FORMULATIONS

(71) Applicant: First Time US Generics LLC, Broomall, PA (US)

(72) Inventors: Manesh A. Dixit, Broomall, PA (US); Vaibhav Pawar, Broomall, PA (US); Rushi Patel, Broomall, PA (US); Mineshkumar Patel, Broomall, PA (US)

(73) Assignee: First Time US Generics LLC, Broomall, PA (US)

(21) Appl. No.: 16/110,156

(22) Filed: Aug. 23, 2018

Related U.S. Application Data
(63) Continuation-in-part of application No. PCT/US2017/019869, filed on Feb. 28, 2017.
(60) Provisional application No. 62/464,170, filed on Feb. 27, 2017.
(60) Provisional application No. 62/301,536, filed on Feb. 29, 2016.

(51) Int. Cl. A61K 9/20; A61K 9/00; A61P 25/30

(52) U.S. Cl. CPC A61K 9/2095; A61P 25/30; A61K 9/0056; A61K 9/2059

(57) ABSTRACT

The present disclosure relates to oral, abuse deterrent, edible soft chewable dosage forms for delivery of drugs that are susceptible to abuse to a human or animal subject. The dosage forms are provided as chewable tablets manufactured using a compression (tablet) press. The edible soft chew dosage forms can be administered to subjects that are unable to swallow conventional tablets or capsules whole. One or more abuse deterrent measures in the dosage forms prevent the conversion of the dosage form into a residue or extract suitable for non-oral administration, such as intranasal or intravenous abuse. The present disclosure also relates to processes of preparing the dosage form. Such soft chew dosage forms have hardness less than 2 kilopond, preferably less than 1 kilopond, more preferably no measurable hardness when measured with tablet hardness tester and friability less than 1%, preferably less than 0.5%, more preferably less than 0.1% for 100 rotations (per USP); 200 rotations or 300 rotations.

### CROSS-REFERENCE TO RELATED APPLICATIONS

The present application is a continuation-in-part of International Patent Application No. PCT/US2017/019869, filed Feb. 28, 2017, which claims the benefit of and priority from U.S. Provisional Patent Application Nos. 62/301,536, filed Feb. 29, 2016, and 62/464,170, filed Feb. 27, 2017, the entire disclosure of each of which is hereby incorporated by reference herein.

### BACKGROUND OF THE INVENTION

### 1. Field of the Invention

The present invention relates products and processes for the manufacture of soft-chewable tablet pharmaceutical dosage forms, for the oral administration of active pharmaceutical ingredients susceptible to abuse and the prevention of drug abuse by the extraction and administration of the active ingredient by alternative routes.

The present invention includes abuse-deterrent formulations for reducing the potential of abuse/misuse (including accidental overdose, inhalation (e.g., intranasal abuse), parenteral abuse, and oral abuse) of an abuse-potential drugs.

Abuse-potential drug products have been a concern in recent years, because prescriptions for such abuse-potential drug products have seen a steady increasing along with a significant escalation in their abuse and misuse. In the United States, deaths caused by prescription drug abuse are greater than deaths associated with illegal drugs like heroin and cocaine together. Also, the abuse of prescription drug products is causing a significant strain and cost to health care system.

Addicts and recreational drug users may abuse such abuse-potential drug products by a pulverizing a tablet or chemically extracting the active agent for use with alternative routes of administration. Commonly used methods include parenteral (e.g., intravenous injection, where the drug is crushed and extracted or melted and the contents of a dosage unit then injected), intranasal (e.g., snorting, where the drug is inhaled through the nose in powdered form), and episodic or repeated oral ingestion of crushed product, where the drug product is chewed to increase the surface area and permit rapid release of the drug substance. All of these strategies are intended to more efficiently get the abuse-potential drug (active) into the CNS, in terms of increasing the total amount of drug ingested, peak concentration of drug, or time to peak concentration of drug (i.e., more rapid onset of action).

Generally, the oral route is preferred by non-experienced and occasional abusers while seasoned or more experienced abusers prefer an injection route. However, the highest mortality and/or severe abuse related complications are associated with the abuse associated with parenteral and nasal routes.

A number of reported cases of abuse-potential drug product (e.g. opioids) toxicity are a result of inadvertent or unintentional medical use of abuse-potential drug products. It is not uncommon for patients who have difficulty swallowing to crush the contents of tablets and swallow the contents with liquids or on soft food. In the case of most immediate release formulations, this generally produces no significant harm, with marginally higher peak concentrations (Cₘₐₓ) and time to peak concentrations (Tₘₐₓ). However, in the case of controlled-release oral dosage formulations, crushing an oral solid dosage form may destroy the controlled release mechanism and result in a rapid surge of drug into the bloodstream; for instance, an entire twelve (12) or twenty-four (24) hour drug supply being released immediately, which can produce toxic effects. For this reason, such controlled-release drug products available for sale in the United States usually carry an appropriate warning(s) instructing the prescriber and patient not to crush or otherwise tamper with the oral solid dosage form.

There is growing recognition in the medical community that large numbers of patients suffer from under-treatment of their medical condition when the treatment involves the use of psychoactive drugs—particularly drugs which tend to diverted and abused.

Scheduling of abuse-potential drug products has also impacted the quality of physician treatment. Fearful of being accused of permitting and/or promoting drug abuse and/or overuse, many physicians will prescribe suboptimal doses of abuse-potential drug products to and/or less effective, non-abuse-potential drug products to their patients.

Thus, there is a need for abuse deterrent methods to protect both medical and non-medical users of abuse-potential drugs from improper use (i.e., abuse), including intentional and unintentional toxicity, without adding unnecessary risk or harm to either group.

### 2. Discussion of the Prior Art

One way of providing safer dispensing of such abuse-potential drug products is to develop drug formulations with design features that prevent or deter abuse (commonly referred to as abuse-deterrent formulations).

The United States Food and Drug Administration (“FDA”) has issued draft guidance for industry related to formulations having abuse deterrent properties entitled “Guidance for Industry: Abuse Deterrent Opioids-Evaluation and Labeling” (U.S. Department of Health and Human Services, FDA, CDER, April 2015). These guidelines separate abuse deterrent formulations into six categories, including: physical/chemical barriers; agonist/antagonist combinations; aversion; delivery system; prodrug; and combinations of the foregoing. In order to characterized as an abuse deterrent formulation, the formulation has to conform to at least one of the six FDA categories.

Adding physical barriers to solid oral dosage forms is one method to prevent abuse based on chewing, pulverizing, cutting, grating, or grinding. Physical barriers prevent or reduce abuse/misuse by making it more difficult to breakdown the dosage form. For example, abuse deterrent dosage forms including physical barriers can make it harder to grind the dosage form, extract the active therefrom, or both, which prevents non-oral administration, such as intranasal or intravenous. One of the ways to introduce the physical barrier is to consolidate one of the physical properties of the oral dosage forms, for example, increase the tablet hardness by use of injection molding technique to resist the abuse by chewing, pulverizing, cutting, grating or grinding. Such attempts to affect the physical attributes of dosage form require significant investment in specialty materials, equipment and complex technologies that are not traditionally used in pharmaceutical manufacturing processes.

Chewable pharmaceutical dosage units, such as chewable tablets and soft-chewable tablets, are known and have been commercialized for pediatric, geriatric and involuntary patient populations. Such dosage forms have also been used for subjects that, by instinct, will not accept the medication meant to be swallowed (e.g., animals). Chewable tablets are also useful with competent patients as an alternative to tablets or capsules that must be swallowed whole. The formulation of a drug into a chewable dosage form can increase patient acceptance of a medication in patients that resist or are unable to swallow conventional tablets or capsules.

Conventional dosage forms, such as chewable compressed tablets, using conventional ingredients, can make the tablet gritty or otherwise unappealing to many patients.

Traditionally, tablets compressed on a compression machine are formulated and processed so the tablets have hardness of more than three kiloponds (3 kp). Tablets having lower hardness levels are discouraged in the prior art to keep the tablet friability acceptable.

A process for manufacturing soft-chewable dosage form for drug delivery is described in U.S. Pat. No. 6,387,381. It discloses a soft-chewable medication vehicle for drug delivery of an active ingredient to animal or human subjects, not containing ingredients of animal origin, without use of heat and without addition of water. The formed mixture was formed into individual chunks using a Formax F6™ molding machine with dies for production of chunk-like shapes, and packaged for storage.

Machines for the production of molded food patties have been described to be useful for the manufacturing of soft-chews for administration to non-human animals. Such machines are molding machines that have been originally developed for use in producing molded food products, for example the Formax F6™ molding machine made by the Formax Corporation.

The use of extruders, forming machines and rotary molding machines exhibit problems associated with the weight and physical forms of a final dosage form. Moreover, the use of such technologies may require conditioning of the final dosage form (e.g. drying or curing final formed structure) for consolidation of shape and structure of formed structure.

Tamper-resistant delivery systems using gelling agents in a drug delivery forms like conventional hard tablets and/or capsules are known in the art. When such a dosage form is dissolved in a small amount of water, instead of a solution, a viscous gel that cannot be injected may be formed. The gel prevents the drug from being removed by cold water extraction, because the gel retains the drugs together when extraction is attempted.

Several attempts have been made in the prior art to provide an abuse deterrent dosage form. For instance, an abuse deterrent dosage form with controlled-release for once daily use is known whereby abuse deterrent potential is built-in by increasing the breaking strength of the dosage from 500 Newtons (N) up to preferably 1000 Newtons (N). An abuse deterrent dosage form of opiod analgesics is known whereby abuse-potential for parental, intranasal and/or oral abuse-potential is reduced by incorporation of gel forming polymer to reduce extraction potential, a surfactant to reduce intranasal abuse-potential and emetic agent to reduce oral abuse-potential respectively. Also, an abuse deterrent oral dosage form is known that includes a gelling agent in the formulation composition that reduces extraction potential in aqueous or organic solvents.

### SUMMARY

The following brief summary is provided to indicate the nature of the subject matter disclosed herein. While certain aspects of the present invention are described below, the summary is not intended to limit the scope of the present invention.

The present invention overcomes the disadvantages and shortcomings of known chewable dosage forms by providing a simplified manufacturing process for soft-chewable dosage unit formulations comprising a highly palatable composition to patients, which is formed by conventional compression techniques using conventional pharmaceutical equipment, such as a rotary tablet press.

The soft-chewable formulations are prepared according to methods conventional in the art, such as wet or dry granulation processes.

One aspect of the present invention relates to oral, immediate and/or controlled-release, abuse deterrent soft chewable dosage forms containing an active ingredient representative of active drug substances susceptible to abuse.

An active pharmaceutical ingredient for use in the process or product according to the current invention (or active ingredient, or pharmaceutically active agent or pharmaceutically acceptable active ingredient) is a substance used in a pharmaceutical dosage form, intended to furnish pharmacological activity or to otherwise have direct effect in the diagnosis, cure, mitigation, treatment or prevention of disease, or to have direct effect in restoring, correcting or modifying physiological functions in a patient population (humans or animals).

Any pharmaceutically active ingredient or nutritional agent may be provided in the process of the invention and in the product according to the invention. Those of ordinary skill in the pharmaceutical arts will be familiar with the identity of such active ingredients which may include antibiotics, analgesics, antiviral, antifungal, anti-parasitic, hormones, anti-inflammatory (including nonsteroidal anti-inflammatory), steroids, behavior modifiers, vaccines, antacids, laxatives, anticonvulsants, sedatives, tranquilizers, antitussives, antihistamines, decongestants, expectorants, appetite stimulants and suppressants, cardiovascular drugs, minerals and vitamins along with other supplement and nutraceutical agents.

The texture of a chewable dosage unit form is an important factor in the acceptance of oral dosage forms by patients in need of medication. Soft-chewable tablet dosage units, having a soft texture, pleasant mouthfeel, and palatable taste with adequate flavoring agents, provide a solution to such problems. In addition, these features can address the problem of the disagreeable taste of many active pharmaceutical ingredients. Appropriate chewable dosage form can also address texture problems caused by dry dusty, granular, and pulverant properties of many pharmaceutical ingredients.

Dosage forms of the present invention include palatable, soft-chewable pharmaceutical compositions for oral administration to an involuntary subject population (e.g., very young children, senile patients, animals, etc.) that includes a therapeutically effective amount of a pharmaceutically active ingredient susceptible to abuse, in an immediate or controlled release form, and a palatability improving agent in an amount sufficient make the pharmaceutical composition palatable to the subject population. As used herein, the phrase “involuntary subject population” is defined as patients who cannot be conventionally instructed to chew and/or swallow conventional hard chew tablets or capsules.

Preferably, the soft chew dosage forms of the present invention have a soft texture and palatable taste with adequate flavoring to increase palatability and chewy mouthfeel can provide an excellent alternative to incorporate one or many physical barriers to prevent abuse of the susceptible active drug substance. Such abuse deterrent soft chews can be manufactured on large scale in traditional pharmaceutical set-up using most commonly used equipment such as a rotary (tablet) compression press.

A soft-chewable pharmaceutical dosage unit is a solid pharmaceutical dosage unit at room temperature that has low hardness and higher moisture content than a conventional tablet or hard chewable tablet. The dosage unit may be designed to be chewed and swallowed by a human or an animal. Such a dosage unit exhibits a plastic rheological behavior and can be formed by many manufacturing processes described in prior art into many different shapes. A soft-chewable pharmaceutical dosage unit after forming should be dimensionally stable. The ingredients of such a soft-chewable pharmaceutical dosage unit may be of pharmaceutical grade.

A semi plastic oral dosage form unit has a soft texture and hardness such that the unit is intended to be chewed and swallowed. The texture of the unit is such that it does not appreciably dissolve in the mouth. A semi plastic oral dosage form unit is formed by compression on rotary tablet press and exhibits hardness of less than 2 kilopond, preferably less than 1 kilopond, and more preferably has no measurable hardness when measured with a tablet hardness tester, which has excellent flexibility, is breakage and chip resistant and yet may be easily chewed and swallowed by human or animal.

Dosage forms of the present invention preferably have a hardness of less than about two kiloponds (2 kp) when tested with tablet hardness tester. More preferably, dosage forms of the present invention have a hardness of less than about one kilopond (1 kp) when tested with tablet hardness tester. Even more preferably, dosage forms of the present invention have no measurable hardness when tested with tablet hardness tester. Despite the low hardness, dosage forms of the present invention have a friability of less than about one percent (1.0%), or less than about one-half percent (0.5%), or less than about one-tenth percent (0.1%), at one-hundred (100) rotations, two-hundred (200) rotations or three-hundred (300) rotations (per USP). The abuse deterrent soft chew tablets of the current invention are chewy and do not leave gritty feeling commonly associated with the tablet compressed on the tablet press in the mouth.

Dosage forms of the present invention may include abuse-deterrent features whereby the release of the abuse-potential drugs through nasal insufflation (i.e., “snorting”) is hindered. For instance, dosage forms of the present invention may include an effective amount of one or more gelling agent(s) along with lipid components to provide both the rapid gelling and nasal irritation. Dosage forms of the present invention may also include surfactants (e.g., sodium lauryl sulfate), which can cause nasal irritation if snorted and thus deter nasal insufflation. Dosage forms of the present invention may inhibit manipulation by grinding or pulverizing using common equipment, such as a coffee grinder. The formulations of the present invention can deter abuse by limiting the particle size to which the formulation may be ground. The formulation prevents the dosage form or at least substantial portions of the dosage form, from being ground in particles having a particle size of about five-hundred micrometers (500 μm) or less that may pass through the mucus membranes of the nasal cavity.

Dosage forms of the present invention may include abuse-deterrent features whereby extraction of an active drug substance for repurposing as an injectable drug is prevented. For instance, gelling agents can be used to prevent extraction of an active drug substance by water or alcohol for repurposing as an injectable drug. Deterring injection of an extracted active substance by common solvents (e.g., cold water or aqueous ethanol) can also be accomplished according to the present invention by means of visual deterrence.

Dosage forms of the present invention may include abuse-deterrent features that prevent or hinder destruction of the release mechanism. For instance, in one embodiment of the present invention, an oral soft chewable tablet dosage form which is either immediate release or controlled release is provided where crushing the dosage form does not destroy the release mechanism and results in identical release pattern of drug into the bloodstream. Such semi plastic oral dosage form units having a soft texture and no hardness are intended to be chewed and swallowed. Irrespective of whether the dosage form is swallowed in entirety or chewed, it will result in identical patterns for total amount of drug in bloodstream, peak concentration of drug and time to peak concentration of drug.

Dosage forms of the present invention may include a pharmaceutically active ingredient that is conjugated with other ingredients that reduce the abuse-potential inhibit its interactions with other excipients or with the environment, or to promote the chemical stability, affect solubility, modulate bioavailability, or improve the palatability of such abuse-potential pharmaceutically active agent, such as cyclodextrins, surfactants, solubility or bioavailability modulators, etc., Similarly, the pharmaceutically active ingredient may be incorporated into a novel drug delivery system, such as microspheres, microcapsules, liposomes, niosomes, nanoparticles, microemulsions, or nanoemulsions to protect the drug or permit organ targeting in addition to affect the abuse-potential.

### BRIEF DESCRIPTION OF THE DRAWING FIGURES

Preferred embodiments of the invention are described in detail below with reference to the attached figures, wherein:

FIG. 1A is a photograph of tablets of example A;

FIG. 1B is a photograph of tablets of example B;

FIG. 1C is a photograph of tablets of example C;

FIG. 1D is a photograph of tablets of example D;

FIG. 1E is a photograph of tablets of example E;

FIG. 1F is a photograph of tablets of example F;

FIG. 2A is a photograph of ground tablets of Example A, ground for 2 minutes (120 seconds) in a coffee grinder;

FIG. 2B is a photograph of ground tablets of Example C, ground for 60 seconds in a coffee grinder;

FIG. 2C is a photograph of ground tablets of Example F, ground for 2 minutes (120 seconds) in a coffee grinder;

FIG. 3A shows tablets of Example A after five (5) minutes of being submerged in ten milliliters (10 ml) of five percent (5%) ethyl alcohol solution at twenty-five degrees Celsius (25° C.), with the intact tablet being shown in the beaker on the left and the crushed tablet being shown in the beaker on the right;

FIG. 3B shows tablets of Example B after five (5) minutes of being submerged in ten milliliters (10 ml) of forty percent (40%) ethanol solution at twenty-five degrees Celsius (25° C.), with the intact tablet being shown in the beaker on the left and the crushed tablet being shown in the beaker on the right;

FIG. 3C shows tablets of Example C after five (5) minutes of being submerged in ten milliliters (10 ml) of ninety-nine percent (99%) isopropanol solution at twenty-five degrees Celsius (25° C.), with the intact tablet being shown in the beaker on the left and the crushed tablet being shown in the beaker on the right;

FIG. 3D shows tablets of Example D after five (5) minutes of being submerged in ten milliliters (10 ml) of seventy percent (70%) isopropanol solution at twenty-five degrees Celsius (25° C.), with the intact tablet being shown in the beaker on the left and the crushed tablet being shown in the beaker on the right;

FIG. 3E shows tablets of Example E after five (5) minutes of being submerged in ten milliliters (10 ml) of water at twenty-five degrees Celsius (25° C.), with the intact tablet being shown in the beaker on the left and the crushed tablet being shown in the beaker on the right;

FIG. 4A shows tablets of Example A after thirty (30) minutes of being submerged in ten milliliters (10 ml) of five percent (5%) ethyl alcohol solution at twenty-five degrees Celsius (25° C.), with the intact tablet being shown in the beaker on the left and the crushed tablet being shown in the beaker on the right;

FIG. 4B shows tablets of Example B after thirty (30) minutes of being submerged in ten milliliters (10 ml) of forty percent (40%) ethanol solution at twenty-five degrees Celsius (25° C.), with the intact tablet being shown in the beaker on the left and the crushed tablet being shown in the beaker on the right;

FIG. 4C shows tablets of Example C after thirty (30) minutes of being submerged in ten milliliters (10 ml) of ninety-nine percent (99%) isopropanol solution at twenty-five degrees Celsius (25° C.), with the intact tablet being shown in the beaker on the left and the crushed tablet being shown in the beaker on the right;

FIG. 4D shows tablets of Example D after thirty (30) minutes of being submerged in ten milliliters (10 ml) of seventy percent (70%) isopropanol solution at twenty-five degrees Celsius (25° C.), with the intact tablet being shown in the beaker on the left and the crushed tablet being shown in the beaker on the right;

FIG. 4E shows tablets of Example E after thirty (30) minutes of being submerged in ten milliliters (10 ml) of water at twenty-five degrees Celsius (25° C.), with the intact tablet being shown in the beaker on the left and the crushed tablet being shown in the beaker on the right;

FIG. 5A shows tablets of Example A after sixty (60) minutes of being submerged in ten milliliters (10 ml) of five percent (5%) ethyl alcohol solution at twenty-five degrees Celsius (25° C.), with the intact tablet being shown in the beaker on the left and the crushed tablet being shown in the beaker on the right;

FIG. 5B shows tablets of Example B after sixty (60) minutes of being submerged in ten milliliters (10 ml) of forty percent (40%) ethanol solution at twenty-five degrees Celsius (25° C.), with the intact tablet being shown in the beaker on the left and the crushed tablet being shown in the beaker on the right;

FIG. 5C shows tablets of Example C after sixty (60) minutes of being submerged in ten milliliters (10 ml) of ninety-nine percent (99%) isopropanol solution at twenty-five degrees Celsius (25° C.), with the intact tablet being shown in the beaker on the left and the crushed tablet being shown in the beaker on the right;

FIG. 5D shows tablets of Example D after sixty (60) minutes of being submerged in ten milliliters (10 ml) of seventy percent (70%) isopropanol solution at twenty-five degrees Celsius (25° C.), with the intact tablet being shown in the beaker on the left and the crushed tablet being shown in the beaker on the right;

FIG. 5E shows tablets of Example E after sixty (60) minutes of being submerged in ten milliliters (10 ml) of water at twenty-five degrees Celsius (25° C.), with the intact tablet being shown in the beaker on the left and the crushed tablet being shown in the beaker on the right;

FIG. 6 is a table showing the quantitative formulations of the dosage forms of Examples A-E;

FIG. 7 is a table showing the product characterization of the dosage forms from Examples A-F;

FIG. 8 is a table showing the results of the tablet grinding study; and

FIG. 9 is a table showing the results of product dissolution study of the dosage form of Example G and an Adderall XR 10 mg capsule.

The figures do not limit the present invention to the specific embodiments disclosed and described herein. The emphasis instead being placed upon clearly illustrating the principles of the preferred embodiment.

### DETAILED DESCRIPTION OF THE PREFERRED EMBODIMENTS

The present invention is susceptible of embodiment in many different forms. While the figures show, and the specification describes, certain preferred embodiments of the invention, it is to be understood that such disclosure is by way of example only. There is no intent to limit the principles of the present invention to the particular disclosed embodiments.

### Definitions

The term “abuse-potential drug” or “abuse-potential pharmaceutical active” means an active pharmaceutical ingredient with an approved pharmaceutical use, but which is subject to abuse. Examples of abuse-potential drugs include narcotic pain relievers, sleeping aids, and anxiolytics. Only orally active and orally administered drugs are within the scope of this disclosure.

The terms “abuse,” “drug abuse,” or “drug product abuse,” in the context of the present invention, refers to a drug which is subject to inappropriate, unapproved, or illegal uses, such as intermittent use, recreational use, and chronic use of abuse-potential drugs alone or in conjunction with other drugs. The term “abuse” also means use in quantities or by methods and routes of administration that do not conform to standard medical practice or are not approved or intended by the manufacturer of the product. Such uses are outside the scope of the specific instructions for use provided by a qualified medical professional, or outside the supervision of a qualified medical professional, outside the approved instructions on proper use provided by the drug's legal manufacturer.

The term “tampering” means any manipulation by mechanical, thermal and/or chemical means which changes the physical or chemical properties of the dosage form. For example, “tampering” includes liberating the abuse-potential pharmaceutical active for immediate release to make it available for inappropriate use such as administration by an alternate route (e.g., injection or inhalation). “Tampering” may also include crushing, shearing, grinding, mechanical extraction, solvent extraction, solvent immersion, combustion, heating or any combination thereof.

The term “abuse-resistant,” “abuse-deterrent,” “tamper resistant,” and “deter abuse” (as well of the words “resist” or “deter” when applied to abuse-potential pharmaceutical active) are used interchangeably in the context of the present invention and include pharmaceutical compositions and that resist, deter, discourage, diminish, delay and/or frustrate the: (a) intentional, unintentional or accidental physical or chemical manipulation or tampering of the dosage form (e.g., crushing, shearing, grinding, chewing, dissolving, melting, needle aspiration, inhalation, insufflation, extraction by mechanical, thermal and chemical means, and/or filtration); (b) intentional, unintentional or accidental use or misuse of the dosage form outside the scope of specific instructions for use provided by a qualified medical professional, outside the supervision of a qualified medical professional and outside the approved instructions on proper use provided by the drug's legal manufacturer (e.g., intravenous use, intranasal use, inhalational use and oral ingestion to provide high peak concentrations); (c) intentional, unintentional or accidental conversion of an controlled release dosage form of the invention into a more immediate release form; (d) intentional and iatrogenic increase in physical and psychic effects sought by recreational drug users, addicts, and patients with pain who have an addiction disorder; attempts at surreptitious administration of the dosage form to a third party (e.g., in a beverage); (e) intentional, unintentional or accidental attempts at otherwise changing the physical, pharmaceutical, pharmacological and/or medical properties of the dosage form from what was intended by the manufacturer.

As used herein with respect to abuse-potential drug dosage forms of the present invention, the terms “oral,” “oral dosage form,” “oral pharmaceutical dosage form,” “oral administration,” and “oral route” refer to any method of administration involving contact with the mouth and oral mucosa, including the ingestion of intact drugs. Particularly preferred embodiments involve oral ingestion of abuse-potential pharmaceutical actives.

A “soft chewable pharmaceutical dosage unit,” “soft chewable tablet,” or “soft chew” is a solid pharmaceutical dosage unit at room temperature that has low or preferably no hardness when measured with traditional hardness testers and higher moisture content than a conventional tablet or hard chewable tablet. The dosage unit may be designed to be chewed and swallowed by a human or an animal. Such a dosage unit exhibits a plastic rheological behavior and can be formed by many manufacturing processes described in prior art into many different shapes. A soft-chewable pharmaceutical dosage unit after forming should be dimensionally stable. The ingredients of such a soft-chewable pharmaceutical dosage unit may be of pharmaceutical grade.

The soft tablet dosage forms of the present invention are also termed “semi-plastic.” As used herein, “semi-plastic” means that the dosage forms are deformable by gentle pressure but returns to its original shape (or its approximate original shape) when the pressure is released.

By the term “active ingredient” or “active agent,” it is meant an active pharmaceutical ingredient (i.e., a drug). An active pharmaceutical ingredient may include any approved or experimental drug. In an embodiment, the drug is susceptible to abuse. By “approved,” it is meant that the drug is approved for human or veterinary use by a regulatory agency in any country that makes such drug approvals.

The terms “drug,” “drug substance,” “substance,” “therapeutic agent,” “pharmacological agent,” “pharmaceutical agent,” and “active agent” are used interchangeably and are intended mean any therapeutically active substance which is delivered to a living organism to produce a desired, usually beneficial effect. This includes therapeutic agents in all of any therapeutic areas.

The term “fluid” refers to a material that is flowable or malleable. A fluid material may be a viscous liquid, with a viscosity comparable to, for example, water, vegetable oil, honey, or peanut butter.

The term “immediate release abuse-potential drug” for purposes of the present invention is an abuse-potential pharmaceutical active for oral administration in a dosage form which formulated to release the active drug from the dosage form immediately (i.e., without an attempt to delay or prolong the release of the active drug from the dosage form, as is the case for extended release dosage forms).

The terms “controlled release” “extended release,” “sustained release,” “modified release,” “delayed release,” and the like are used interchangeably herein. Such products provide a longer duration of action than conventional immediate release formulations of the same drug. Controlled release dosage forms of the present invention release active drug from the oral dosage form at slower rate than immediate release formulations. In some instances, controlled release dosage forms release abuse-potential pharmaceutical active at such a rate that blood (e.g., plasma) concentrations (levels) or therapeutic effects are maintained within the therapeutic range (above the minimum effective therapeutic concentration) but below toxic levels for intended duration (e.g., over a period of 1 to 24 hours). Notwithstanding the foregoing, in some preferred embodiments, the controlled release formulations of the present invention provide therapeutic effects for a duration that is longer or substantially longer than the duration of meaningful or detectable plasma concentrations of abuse-potential pharmaceutical active. Controlled release dosage forms may be administered around the clock on a scheduled or time contingent basis, or on an as needed basis.

A “functional coating” means an extended-release coating, delayed-release coating, controlled-release coating, or a combination thereof.

An “aversive agent” is a substance added to the product to produce an unpleasant effect if the dosage form is manipulated or is used at a higher dosage than directed. For example, the formulation can include a substance irritating to the nasal mucosa if ground and snorted.

### Dosage Forms

One aspect of the present invention relates to a solid, abuse-resistant, soft chewable or semi-plastic oral dosage form system including at least one pharmaceutically active drug substance with abuse-potential. Preferably, the solid, abuse-resistant, soft chewable or semi-plastic oral dosage forms of the present invention are obtained by compression on a rotary tablet press. Preferably, the solid, abuse-resistant, soft chewable or semi-plastic oral dosage forms of the present invention exhibit a hardness of less than two kiloponds (2 kp) when measured on tablet hardness tester. More preferably, the solid, abuse-resistant, soft chewable or semi-plastic oral dosage forms of the present invention exhibit a hardness of less than one kilopond (1 kp) when measured on tablet hardness tester. Even more preferably, the solid, abuse-resistant, soft chewable or semi-plastic oral dosage forms of the present invention exhibit no hardness when measured on tablet hardness tester. Preferably, the solid, abuse-resistant, soft chewable or semi-plastic oral dosage forms of the present invention have a friability of less than about 1% at 100 rotations.

Aspects of the present invention relate to dosage forms having abuse deterrent features. For instance, one aspect of the present invention relates to soft chewable or semi-plastic oral dosage form tablets that are resistant being ground or pulverized in a suitable powder for snorting (intranasal inhalation or insufflation).

Dosage forms of the present invention may include abuse deterrent features that make it difficult to grind or pulverize the dosage form into a dry powder with particle size of less than five-hundred micrometers (500 μm). Dosage forms of the present invention may also retain a slightly oily or moist texture, and, because they are soft, are not easily crushed or ground. Additionally, because of the moist texture, they are not easily inhalable.

Dosage forms of the present invention may include abuse deterrent features that make it difficult to separate from the active abuse-susceptible agent by ordinary consumers. For instance, dosage forms of the present invention may include auxiliary additives that prevent or deter administration of the abuse-susceptible agent by alternative routes such as by injection or concentration into more potent oral dosage forms. For example, a gelling agent makes the preparation of an injectable solution very difficult.

Dosage forms of the present invention may include abuse deterrent features that chemically interfere with abuse of the drug. For instance, embodiments of the present invention may include one or more auxiliary substances selected from the group consisting of at least one substance which irritates the nasal passages and/or pharynx (e.g., sodium lauryl sulfate); at least one viscosity-increasing agent, which, with the assistance of a necessary minimum quantity of an aqueous liquid forms a gel which remains visually distinguishable when introduced into a further quantity of an aqueous liquid (e.g., poly ethylene oxide); at least one antagonist for pharmaceutical active agent with potential for abuse; at least one emetic (e.g., capsaicin); at least one dye as an aversive agent (e.g., FD&C Blue 1); and at least one bitter substance (e.g., quinine sulfate).

With any of the auxiliary substances, altering the dosage form to change the route of administration is more difficult. For example, a nasal irritant can be selected that has no effect when the dosage form is normally chewed and swallowed, but that makes conversion of the dosage form to an inhalable powder undesirable, since intranasal administration (snorting) will be irritating. Likewise, a viscosity increasing agent makes chemical extraction for injection difficult, since the extract would be more viscous than normal and difficult to use with a syringe.

### Manufacture

Dosage forms of the present invention are preferably formed by first forming a soft chew mass. The soft chew mass includes various excipients including lipid and dry ingredients, granulation ingredients (intra-granulation ingredients), extra-granulation ingredients, and active pharmaceutical substances. The soft chew mass is then compressed using a rotary tablet press. The inventors have discovered that by appropriate formulation of the soft chew mass, conventional tablet compression techniques can be used to form very soft tablets. In some embodiments of the present invention, the dosage forms are tablets. Preferably, tablets of the present invention will have a uniform composition.

Dosage forms of the present invention may include abuse-potential active pharmaceutical ingredient in granular form, with the active ingredient being coated, or further coated, with a suitable coating. For example, the coating could be a coating polymer that coats and protects the pharmaceutically active agent, or masks an offensive taste and/or offensive odor. In certain embodiments, the coating may be a functional coating; for example, an extended-release coating, delayed-release coating, controlled-release coating, barrier coating, or a combination thereof.

In certain embodiments of the present invention, the pharmaceutically active ingredient may be an opiod analgesic selected from the group consisting of alfentanil, buprenorphine, butorphanol, carefentanil, codeine, dezocine, diacetylmorphine, dihydrocodeine, dihydromorphine, diprenorphine, etorphine, fentanyl, hydrocodone, hydromorphone, β-hydroxy-3-methylfentanyl, leva α-acetylmethadol, levorphanol, lofentanil, meperidine, methadone, morphine, nalbuphine, oxycodone, oxymorphone, pentazocine, pethidine, prepoxyphene, remifentanil, sufentanil, tilidine, tramadol, and pharmaceutically acceptable salts, esters and prodrugs thereof.

In certain embodiments of the present invention, the pharmaceutically active ingredient may be a hypnotic agent selected from the group consisting of alprazolam, diazepam, flurazepam, loprazolam mexazolam, nitrazepam, barbiturate, chlormethiazole, eszopiclone, ramaelteon, zaleplon, zopiclone, zolpidem, and pharmaceutically acceptable salts, esters and prodrugs thereof.

In certain embodiments of the present invention, the pharmaceutically active ingredient may be an anxiolytic selected from the group consisting of amphetamine, alprazolan, diazepam, lorazepam, medazepam, oxazepam, pentylenetetrazole, and pharmaceutically acceptable salts, esters and prodrugs thereof.

In certain embodiments of the present invention, the pharmaceutically active ingredient may be a CNS stimulant selected from the group consisting of caffeine, theophylline, amphetamine, benzphetamine hydrochloride, dextroamphetamine, dextroamphetamine sulfate, levamphetamine, methamphetamine, methylphenidate, modafinil, pemoline, an sibutramine, and pharmaceutically acceptable salts, esters and prodrugs thereof.

In certain embodiments of the present invention, the abuse-potential active ingredient is mixed with a vegetable oil to form a premix. The vegetable oil may be, for example, soybean oil, olive oil, flaxseed oil, canola oil, or corn oil.

In certain embodiments of the present invention, the abuse-potential pharmaceutically active ingredient is added to the composition by dry blending.

In certain embodiments of the present invention, the abuse-potential pharmaceutically active ingredient may be dissolved, emulsified, or suspended in a non-aqueous solvent before addition. The nutritional or pharmaceutically active ingredient may be soluble, partially soluble, or insoluble in water.

Insufflation (intranasal inhalation or “snorting”) is a common route of abuse for abuse-potential drug products. Oral dosage forms can sometime be abused by crushing tablets to a fine powder that can be inhaled. Current abuse-deterrent formulations have limitations against this abuse. Breaking the tablets using forces greater than five-hundred newtons (500 N) (with traditional “tablet breaking force” definitions) does not address abuse deterrence potential of oral drug products with higher tablet breaking force. Test methods using flat platens to crush the product as a criterion for abuse deterrence called “Tablet Hardness Test” is not meaningful because such drug products can be cut with an edged surface (e.g., scissors or a razor blade) and therefore can potentially be abused, with forces that are substantially lower than what has been reported using the breaking strength test or equivalent (e.g., greater than 500 N). To be attractive for insufflations, crushing a product should yield particles of less than five-hundred micrometers (500 μm) to allow uptake of the active substance though the nasal mucosa. Best way to resist insufflations potential is to formulate such drug product in such a way that breaking or crushing drug product yields less than fifty percent (50%) of the particles being less than five-hundred micrometers (≤500 μm) in size. Grinding can be a better evaluation of the relative resistance of such abuse potential drug products.

The inventors of the present invention have found that by appropriate granulation of pharmaceutical formulations, a soft-chew composition is formed that can be pressed into soft-chew tablets using conventional tablet press equipment. This is distinguished from prior art methods for the manufacture of soft-chew tablets that require expensive and complex molding or extrusion equipment. Accordingly, this invention provides unit dosage forms for the administration of abuse-potential pharmaceutically active agents to humans or animals orally, wherein the dosage forms are soft-chew tablets formed by compression in a tablet press and crushing drug product yields less than fifty percent (50%) particles less than or equal to five-hundred micrometers (≤500 μm) in size; thus, resisting insufflations potential.

An exemplary process for the manufacture of an edible semi-plastic unit oral dosage form resistant to abuse may include the steps of:

a. mixing at least one abuse-potential active ingredient with a lipid in solid and/or liquid form to form a premix;

b. optionally, mixing at least one active ingredient with at least one dry or liquid component to form a fluid premix;

c. blending dry ingredients comprising a bulking agent, a lipid, a flavoring agent, a disintegrating agent, a binding agent, a surfactant, a preservative, a lubricating agent, and an anti-sticking agent, or a mixture thereof, to form a uniform dry ingredient mixture;

d. blending the premix and the uniform dry ingredient mixture to form a granulated compacted soft-chew mass;

e. sifting the granulated compacted soft-chew mass through at least one sifting screen to form uniform granules of the soft-chew mass;

f. adding a lubricant or anti sticking agent to the uniform granules of the soft-chew mass;

g. optionally drying the resulting soft-chew mass at controlled temperature of less than fifty degrees Celsius (50° C.);

h. compressing the resulting soft-chew mass in a tablet press to from soft-chewable tablets.

In certain embodiments of the present invention, a single excipient or group of excipients may have more than one function in the formulation of the dosage form. For example, vegetable oil, maltodextrin, poly(ethylene) oxide and modified corn starch may be present to aid in smooth texture, creamy mouthfeel when chewed and provide stable viscosity in presence of minimal heat, however if drug product is abused by nasal insufflations, same ingredients could also cause nasal discomfort and/or irritation; hence, acting as aversive agents as well.

In certain embodiments of the present invention, the amount of an aversive agent in the dosage form can be a fixed ratio in relation to the amount of abuse-potential pharmaceutical active and/or other ingredients in the dosage form. By appropriately selecting the quantity of the aversive agent in the dosage form, aversive effects can be avoided under conditions of proper medical use (e.g., in accordance with manufacturer prescribing directions). However, under some conditions of abuse (e.g., excessive intake of the dosage form of the present invention) the quantity of aversive agent consumed will exceed the “no effect” or “minimum effect” threshold, thereby producing one or more aversive effects, which may include nausea, emesis, diarrhea, laxation, headache, bitter taste, nasal irritation, oro-mucosal irritation (which can lead to abstinence from the abuse-potential pharmaceutical active of the dosage form).

In certain embodiments of the present invention, the dosage units may include a humectant. A humectant is used to retain moisture in the dosage unit and/or resist formation of fine particles when crushed so that drug product yields less than fifty percent (50%) particles less than or equal to five-hundred micrometers (≤500 μm) in size when crushed or pulverized. A humectant of value in this invention may be selected from polydextrose, sodium hyalarunate, propylene glycol, sodium lactate, etc. Liquid humectants include, but are not limited to, glycols, polyols, sugar alcohols, vegetable oils and mineral oil, hydrogenated vegetable oils, hydrocarbons, triacetin, liquid paraffin, or any combination of any two or more thereof. Other humectants known in the art may also be used.

In certain embodiments of the present invention, the soft-chew tablets may incorporate abuse-deterrent technology, which can include one or more of high-melting-point excipients that resist heating and injecting; taste modifiers that resist covert administration, snorting (ingestion of a powdered material through the nose) and dose dumping (extraction of API from tablets); water insoluble excipients that resist extraction and drink adulteration; waxy excipients that resist snorting; viscosity modifiers that resist dissolution, injection and dose dumping; low-density excipients that resist drink adulteration; and dyes, that resist adulteration.

In certain embodiments of the present invention, the formulation includes a dye. A dye can be useful in deterring abuse by discouraging the abuser from intravenous injection. For example, extraction of the dye along with the active ingredient would result in a colored solution that would discourage the abuser from intravenous injection. Thus, in certain embodiments, the dye reduces abuse by extracting and injecting. The dye may be selected from known dyes suitable for use in pharmaceutical formulations or approved by the FDA for such use. Various FDA approved dyes, lakes and colorants, beverage dyes, lakes and colorants, non-tissue staining beverage dyes, lakes and colorants, fecal discolorants, urine discolorants can be employed.

In certain embodiments of the present invention, the formulation may include an alcohol gelling/thickening agent (may be a gelling or thickening agent known to one skilled in the art for use in pharmaceutical formulations) such as acacia, alginic acid, bentonite, calcium acetate, carbomers, carboxymethylcellulose, ethylcellulose, gelatin, hydroxyethylcellulose, hydroxypropyl cellulose, magnesium aluminum silicate, methylcellulose, poloxamers, polyvinyl alcohol, polyvinyl acetate, polyvinylpyrrolidone, sodium alginate, sorbitol derivatives, tragacanth, and, xanthan gum.

### Hardness Properties

In certain embodiments of the present invention, the soft-chewable tablet maintains a characteristic selected from chewiness, hardness, compression energy, adhesion, cohesiveness, springiness, modulus, and any combination of the foregoing (when measured by the texture analyzer) sufficient to provide a chewable texture.

The tablets of the present invention are preferably soft (which is measured by hardness) and have good friability (which means they will not break under routine storage and usage conditions). The breaking force of tablets is commonly called hardness in the pharmaceutical literature. The term crushing strength is also frequently used to describe the resistance of tablets to the application of a compressive load.

The measure of the mechanical integrity of tablets is their breaking force or hardness, which is the force required to cause them to fail (i.e., break) in a specific plane. Various equipment is used for hardness measurements, for example a Monsanto Hardness Tester, Stokes Hardness tester, Pfizer Hardness Tester, Strong-Cobb Hardness Tester, or Schleuniger Hardness tester. Tablet hardness can be expressed using various units depending on the equipment used for hardness measurement. Typically, tablet hardness is expressed in newtons, pounds, Strong-Cobb units, or kiloponds.

With respect to the exemplarily examples provided herein, tablet hardness was measured using a Schleuniger Hardness tester and the applicable units are kiloponds. This apparatus has two parallel platens between which a tablet is placed. A load is applied and the value of the hardness is measured. The platen faces are polished smooth, precision-ground, and arranged perpendicularly to the direction of movement. Perpendicularity is preserved during platen movement, and the mechanism is free of any bending or torsion displacements as the load is applied. The contact faces are larger than the area of contact with the tablet. With respect to the exemplarily examples provided herein, tablet hardness was measured immediately after a soft chewable dosage form was compressed on a rotary tablet press.

In certain embodiments of the present invention, the chewable formulation of this invention includes dosage units which have hardness of less than two (2) kilopond, preferably less than one (1) kilopond, and more preferably has no measurable hardness when measured with a tablet hardness tester.

In certain embodiments of the present invention, the chewable formulation of this invention includes dosage units with hardness less than three (3.0) Strong Cobb units, preferably less than one and one-half (1.5) Strong Cobb units, or more preferably no measurable hardness when measured with a tablet hardness tester.

In certain embodiments of the present invention, the chewable formulation includes dosage units with hardness less than five (5.0) pound, preferably less than two and one-half (2.5) pound, or more preferably no measurable hardness when measured with a tablet hardness tester.

In certain embodiments of the present invention, the chewable formulation includes dosage units with hardness less than twenty (20.0) newtons, preferably less than ten (10) newtons, or more preferably no measurable hardness when measured with a tablet hardness tester.

Generally, tablets having a hardness of five (5) kilopond or less will have a high rejection rate because of an inability to withstand physical stress experienced during production, packaging, and transport. For such tablets tablet friability is generally between one-tenth percent and one percent (0.1% and 1.0%) when measured according to the United States Pharmacopeia (USP) test. A friability value of about one percent (1%) or less (when measured as per USP test) is desirable for tablets in order for them to withstand the stress of handling during production, packaging, and transport. Generally, tablet hardness and friability are inversely proportional (e.g., as tablet hardness decreases, tablet friability generally increases); however, the inventors of the present invention have unexpectedly found that for soft-chewable tablets with hardness of less than two (2) kilopond have a friability of less than one percent (1%), preferably less than one-half percent (0.5%), more preferably less than one-tenth percent (0.1%) for 100 rotations (per USP); 200 rotations or 300 rotations.

For traditional tablet compression using rotary tablet press, tablet hardness is conventionally 3 kilopond or more. As dosage form size increases, compression force is increased to produce tablet with even higher hardness.

### Grinding Properties

In certain embodiments of the present invention, the soft-chew tablets are resistant manipulation by grinding or pulverizing using common equipment, such as a coffee grinder, which can deter abuse by limiting the particle size to which the formulation may be ground. Preferably, the formulation of the present invention prevents the dosage form, or at least substantial portions of the dosage from, from being ground in particles having a particle size of about five-hundred micrometers (500 μm) or less that, which prevents passage through the mucus membranes of the nasal cavity. For example, using commercially available Mr. Coffee® coffee grinder and with fine pulverizing setting, formulation examples were ground for durations of thirty (30) seconds, one (1) minute, or two (2) minutes. The ground dosage form was then analyzed for particle size. Particle size analysis was conducted utilizing a five-hundred micrometers (500 μm) particle size sieve (35 mesh) and pan. For the purposes of this study, any particle less than five-hundred micrometers (<500 μm) in diameter is considered suitable for intranasal abuse. It is generally accepted that any particle less than five-hundred micrometers (>500 μm) in diameter cannot be sufficiently absorbed by the blood vessels in the nasal passages.

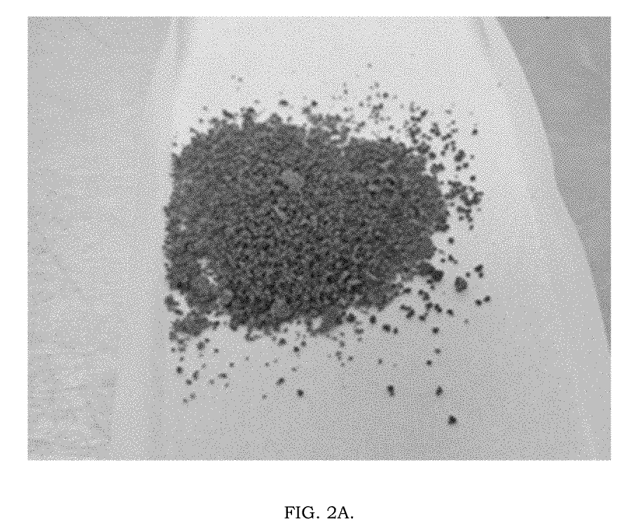

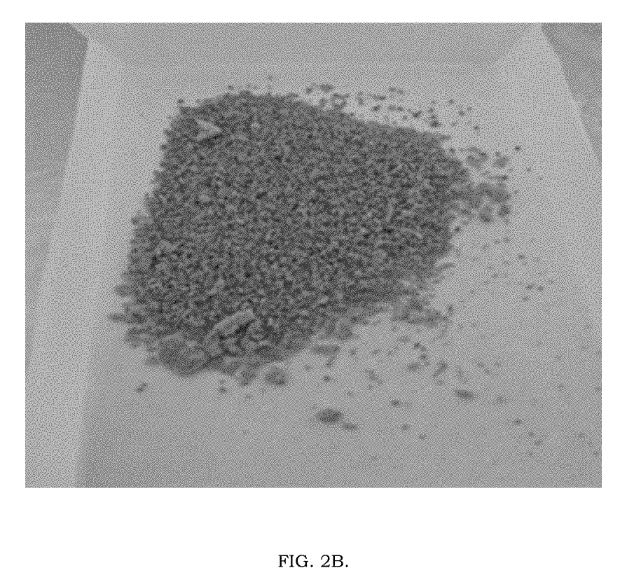

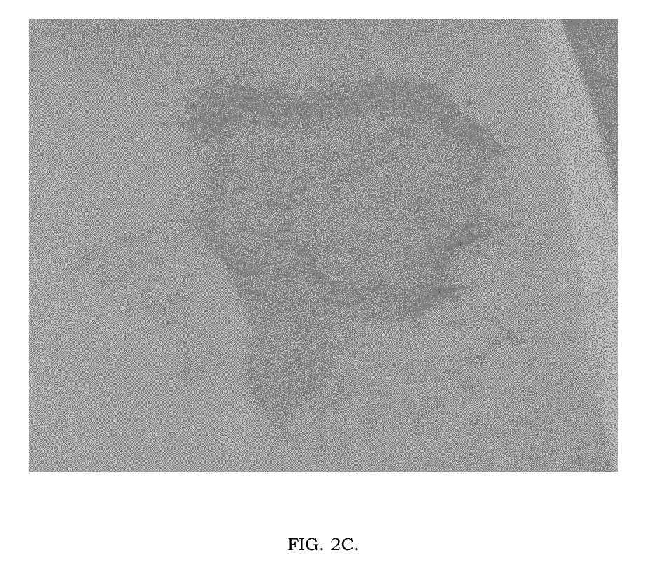

Samples of ground tablets are shown in FIG. 2. FIGS. 2A and 2B show tablets of the present invention having large particle sizes not suitable for nasal ingestion. FIG. 2C is a ground sample of reference Example F, a conventional hard chew formulation. It is readily apparent that the tablet of Example F was pulverized into very small particles, which would be suitable for abuse by nasal inhalation.

### Disintegration

In certain embodiments of the present invention, the soft-chewable tablets disintegrate in less than about fifteen (15) minutes, or less than about thirty (30) minutes, or less than about sixty (60) minutes, according to the USP disintegration test <701> using water as the medium.

In certain embodiments of the present invention, a color is added to the dosage form formulation. An added color can be an abuse deterrent feature. Color may also be an identifying characteristic of commercial drug products. Color can be applied to the dosage form in two ways: dying or coating. High potency alcohol (95%) is an extraction solvent that can be used by abusers for pharmaceutical active ingredients which are insoluble in water or in order to separate the pharmaceutical active ingredients from other water soluble excipients. Dyes or coatings can potentially be used to alter the physical appearance of the extracted solution of drug product. Accordingly, the inclusion of one or more dyes in a drug formulation is one method to render a formulation abuse deterrent. Significant discoloration of an extraction product from a formulation subject to abuse can discourage a potential abuser from using (e.g., injecting or ingesting) the extraction product. A study was conducted to investigate the effect of dyes in the formulations of the present invention. Extraction products from exemplary formulations were visually inspected to observe and demonstrate visual abuse deterrence following extraction.

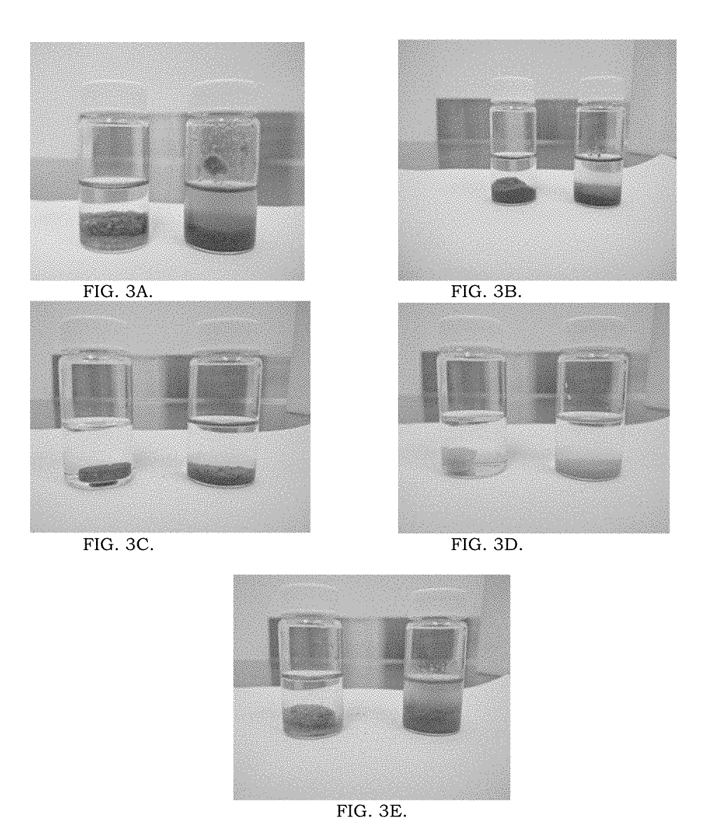

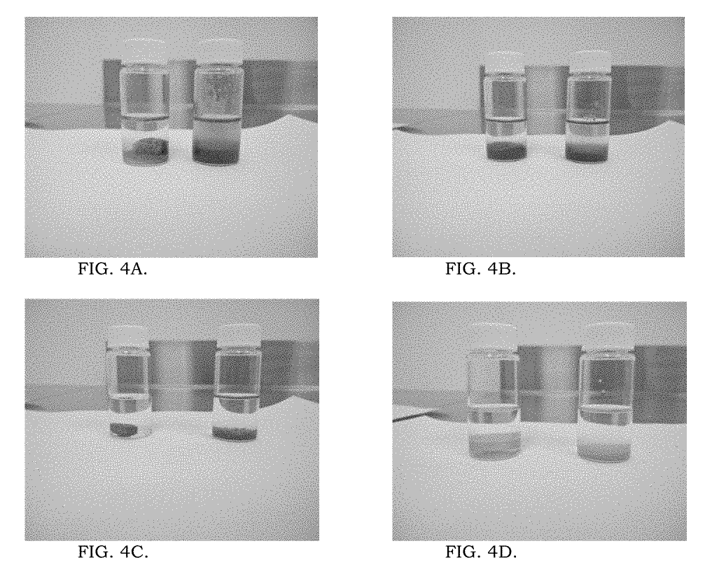

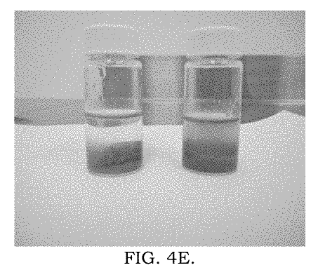

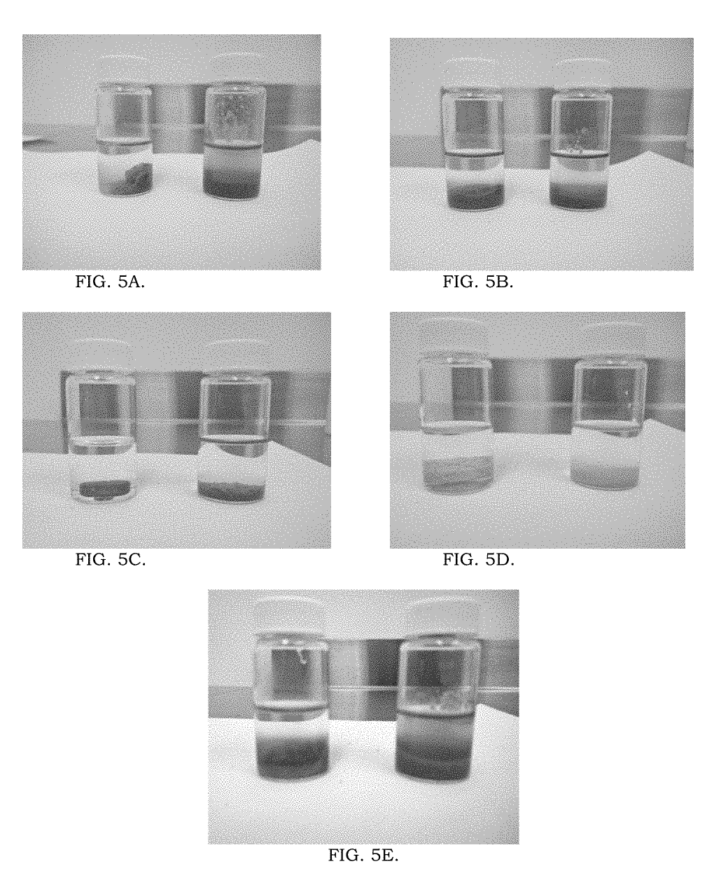

Samples of tablet formulation Examples A-E in 10 ml each of various solvents (5% ethyl alcohol, 40% ethyl alcohol, 99% isopropyl alcohol, 70% isopropyl alcohol, and water) that may be used for extraction by abusers are shown in FIGS. 3, 4, and 5. In each case, an intact tablet and a crushed tablet were added to the solvent and observed after time intervals of five (5), thirty (30), and sixty (60) minutes. Tablets were crushed manually by squeezing them between a person's fingers. These images demonstrate various degree of dark colored dispersion being formed that can be visually unappealing to intravenous drug users. The color was extracted fairly efficiently in water and dilute ethanol. But in more polar solvents, the color was not extracted efficiently, even after 60 minutes. The color was also not extracted efficiently from intact tablets.

The formulation can be formulated with water- and alcohol-soluble dyes to create a dark colored solution upon extraction that can be visually unappealing to intravenous drug users.

### EXAMPLES

Most abuse-potential pharmaceutical actives are controlled drug substances and scheduled and their distribution is tightly controlled because of abuse-potential or risk. Controlled drugs are rated in the order of their abuse risk and placed in Schedules by the Federal Drug Enforcement Administration (DEA). In the examples below, caffeine was selected as a surrogate for abuse-potential drugs because it has similar physicochemical properties as those of abuse-potential drugs. Caffeine is not controlled drug and hence is not rated or scheduled by Federal Drug Enforcement Administration (DEA).

It is necessary to be able to measure resistance or deterrence of the likely routes of abuse in a meaningful and relevant way. The in vitro tests below are provided for illustration of some testing methods and are intended to be non-limiting examples. This invention therefore contemplates the use of test methods other than those specifically disclosed herein, including those which may hereafter become known to the art to perform the necessary functions.

TABLE 1
Physico-chemical properties of various
abuse-potential drugs and Caffeine.

| | Half-Life | | Aq. Solubility |
|---|---|---|---|
| Drug | (h) | pKa | (mg/L)^{a} |
| Codeine | 3 | 9.19 | 577 |
| Propoxyphene | 6-12 | 9.52 | 4 |
| Meperidine | 3-4 | 8.16 | 1110 |
| Hydrocodone | 1-3 | 8.61 | 797 |
| Morphine | 2-4 | 9.12 | 10200 |
| Oxycodone | 3-4 | 8.21 | 5590 |
| Methadone | 15-30 | 9.12 | 6 |
| Hydromorphone | 2-3 | 8.59 | 4390 |
| Oxymorphone | 3-6 | 7.34 | 25600 |
| Fentanyl | 7 | 8.77 | 24 |
| Caffeine | 3-7 | 10.4 | 11000 |

^{a}Predicted value from ALOGPS for the free-base.

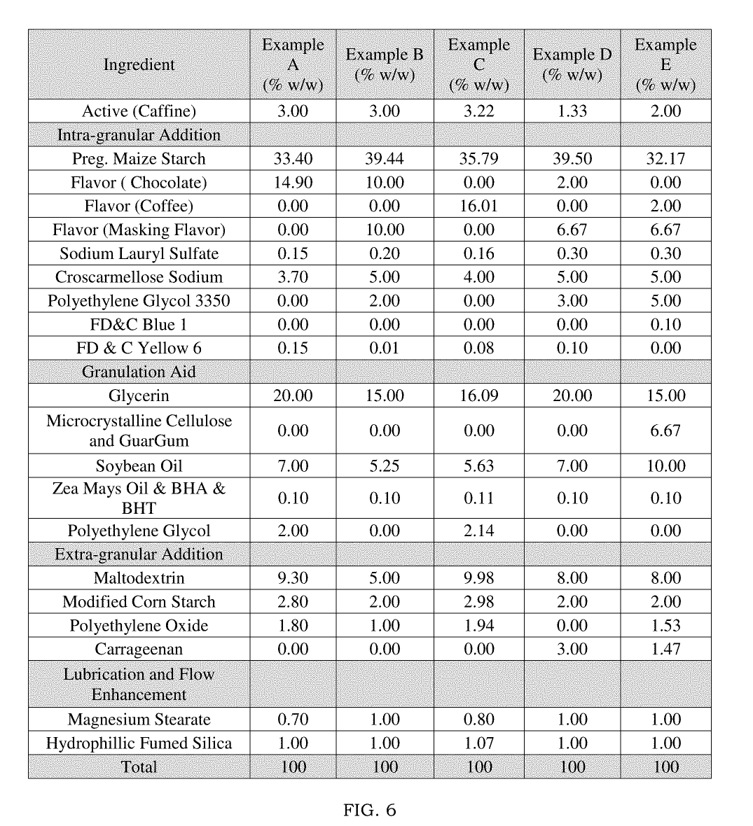

The quantitative formulations for Examples A-E are tabulated in FIG. 6.

### Procedure for Example A, Example C, Example D, and Example E

1. All intra-granular ingredients and active were passed through a sifting screen followed by uniform mixing for approximately five (5) minutes.

2. All extra-granular ingredients were milled through passed through a sifting screen and mixed for approximately five (5) minutes.

3. Glycerin, Zea Mays Oil & BHA & BHT and soybean oil were added to intra-granular blend and mixed thoroughly until uniformly mixed, followed by quick addition of melted polyethylene glycol 3350 followed by uniform mixing to form granulated mass.

4. Granules from step 3 were passed through passed through a sifting screen.

5. The extra-granular blend from step 2 was added to screened granules from step 4, followed by uniform mixing for approximately five (5) minutes.

6. The blended granules from step 5 were passed through passed through a sifting screen.

7. Magnesium stearate and hydrophilic fumed silica were weighed and mixed with a small amount of the granules from step 6 and passed through a sifting screen

8. The milled materials from step 7 were added to granules from step 6 followed by uniform mixing for approximately one to two (1-2) minute(s) to give a soft-chew mass.

9. The soft-chew mass was compressed on a rotary tablet press a using a capsule shaped 17.6 mm×7.5 mm punch.

### Procedure for Example B

1. All intra-granular ingredients and active were passed through a sifting screen followed by uniform mixing for approximately five (5) minutes.

2. All extra-granular ingredients were passed through a sifting screen and mixed for approximately five (5) minutes.

3. Glycerin, Zea Mays Oil & BHA & BHT, and soybean oil were added to the intra-granular blend and mixed thoroughly until uniformly mixed to form a granulate.

4. The granulate from step 3 was passed through a sifting screen.

5. The extra-granular blend from step 2 was added to screened granules from step 4, followed by uniform mixing for approximately five (5) minutes.

6. The blended granules from step 5 were passed through a sifting screen.

7. The requisite quantity of magnesium stearate and hydrophilic fumed silica were weighed and mixed with a small amount of the granules from step 6 and passed through a sifting screen

8. The milled materials from step 7 were added to the granules from step 6 followed by uniform mixing for approximately one to two (1-2) minute(s) to give a soft chew mass.

9. The soft chew mass was compressed on a rotary tablet press a using a capsule shaped 17.6 mm×7.5 mm punch.

### Hard Chew Example F

TABLE 2
Quantitative Formulation for Example F (Hard Chewable Tablets)

| Ingredient | Example E (% w/w) |
|---|---|
| Caffeine | 3 |
| Pharmburst 500 | 0 |
| Advantol 300 | 80 |
| Avicel 101 | 0 |
| Flavor (Chocolate) | 10 |
| Croscarmellose Sodium | 0 |
| Fructose D.C | 6.35 |
| FD & C Yellow 6 Lake (Color) | 0.25 |
| Sodium Stearyl Fumarate | 0 |
| Hydrophilic Fumed Silica | 0 |
| Magnesium Stearate | 0.2 |
| Total | 100 |

### Procedure for Comparative Example F (Not Soft) (Based on Recommendations from Pharma Ingredient Manufacturer Formulary)

1. Prepare a premix by weighing all components (except magnesium stearate and color) individually and screen through a sifting screen and mixing for approximately five (5) minutes.

2. Separately weigh magnesium stearate and FD&C Yellow and blend with a small quantity of the premix from step 1 and pass through a sifting screen and mix for approximately five (5) minutes.

3. Added screened ingredients from step 2 and added to mixed ingredients from step 1 and mixed for uniform mixing for two to three (2-3) minutes.

4. The mass from step 3 was compressed on a rotary tablet press a using a capsule shaped 17.6 mm×7.5 mm punch.

### SUMMARY OF RESULTS

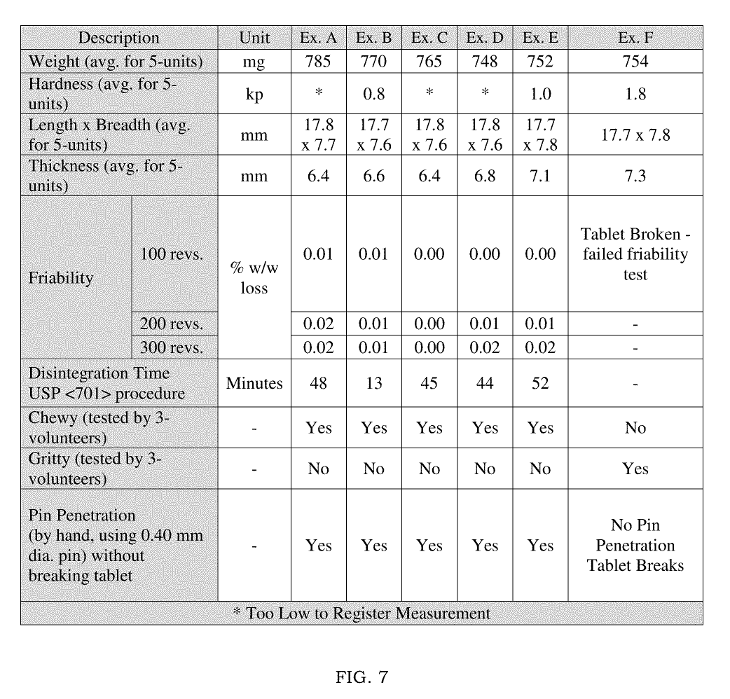

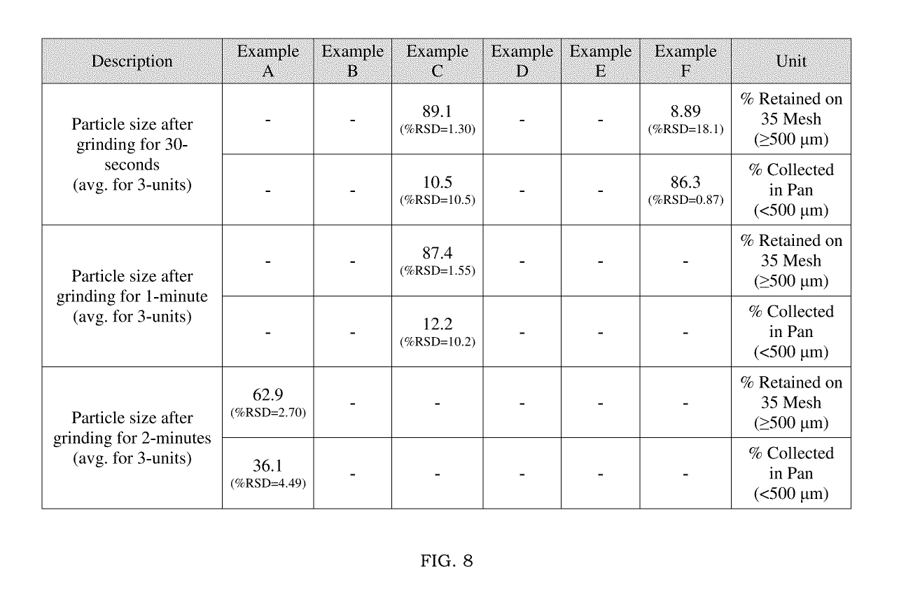

The tablets from examples A-F were tested for the parameters shown in FIGS. 7 and 8.

Each of the examples A-E had a hardness of one kilopond (1.0 kp) or less. The products of examples A-E had good friability and passed a pin penetration test, in which a pin was manually inserted into the tablet. A tablet passed this test if it did not break. The hard chew tablet from example F failed the friability and pin penetration test.

The chewiness and grittiness mouthfeel were tested. The products of examples A-E were chewy and not gritty. The hard chew tablet was not chewy and was gritty.

The grindability of Examples A, C, and F were tested (see FIG. 8). As discussed above, a method of abuse of hard tablets is to pulverize or grind them, which can be done with a simple household coffee grinder.

The tablets of Example F were ground in a household coffee grinder on the finest grind setting for a period of thirty (30) seconds. The ground tablet material was passed through a 35 mesh (500 μm) sieve. Nine percent (9%) of the material was retained on the sieve, and eighty-six percent (86%) passed though. Thus, eighty-six percent (86%) of the tablet from Example F could be ground to a particle size of less than five-hundred micrometers (500 μm) after thirty (30) seconds of grinding, which is small enough for effective insufflation (snorting). Therefore, the vast majority of the material in the hard chew tablet was easily converted to a form suitable for abuse.

The tablets of Example C were ground in a household coffee grinder on the finest grind setting for a period of thirty (30) seconds. The ground tablet material was passed through a 35 mesh (500 μm) sieve. Eighty-nine percent (89%) of the material was retained on the sieve, and ten percent (10%) passed though. Thus, only ten percent (10%) of the tablet from Example C could be ground to a particle size of less than five-hundred micrometers (500 μm) after thirty (30) seconds of grinding. Therefore, relative to Example F tablets, tablets of Example C are much less susceptible to abuse by conversion to a powdered dosage form.

Furthermore, when the tablets of Example C were ground for a period of sixty (60) seconds, the percentage of particles having a size less than five-hundred micrometers (500 μm) increased only by a small amount. More particularly, when ground for one (1) minute, only twelve percent (12%) of the tablet from Example C could was reduced to a particle size of less than five-hundred micrometers (500 μm).

The tablets of Example A were ground in a household coffee grinder on the finest grind setting for a period of two (2) minutes. The ground tablet material was passed through a 35 mesh (500 μm) sieve. Sixty-three percent (63%) of the material was retained on the sieve, and ten percent (36%) passed though.

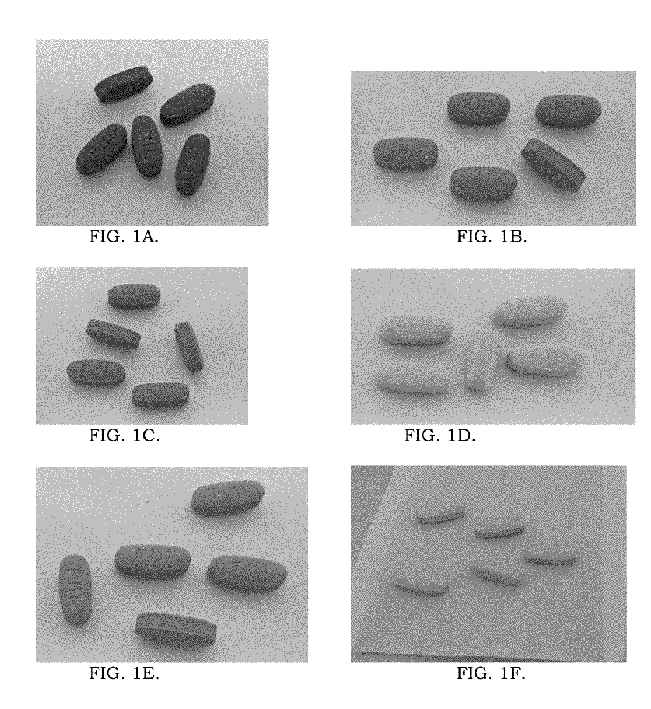

The results of the grinding study show that the soft chew tablets qualify as abuse deterrent because significant quantities of the original tablet material could not be easily pulverized into a powder suitable for insufflation. The ground tablet material produced by grinding samples of tablets of Examples A, C, and F are shown in FIG. 1.

The visual deterrence potential of Examples A-E were each tested using ten milliliters (10 ml) of common household solvents (5% ethyl alcohol, 40% ethyl alcohol, 99% isopropyl alcohol, 70% isopropyl alcohol, and water) that could be used for extraction by abusers. The results are shown in FIGS. 3-5. In each case, an intact tablet and a crushed tablet (manually crushed between fingers) were added to a solvent and observed after time intervals of five (5), thirty (30), and sixty (60) minutes. As shown in FIGS. 3-5, various degrees of dark colored dispersion being formed that can be visually unappealing to intravenous drug users. The water soluble dyes were not extracted even after 60 mins, but in the crushed tablets, water and dilute alcohol yielded a dark color dispersion when compared to intact tablet in same volume of solvent. Certain dyes such as FD&C Blue 1 (Example E) yield a significant dark color that can be visually unappealing as against FD & C Yellow 6 (Examples A-D). This demonstration indicates that the formulation can be formulated with water-soluble and alcohol-soluble dyes to create a dark colored solution upon extraction that can be visually unappealing to intravenous drug users.

### Example G. Amphetamine Soft Chew Formulation

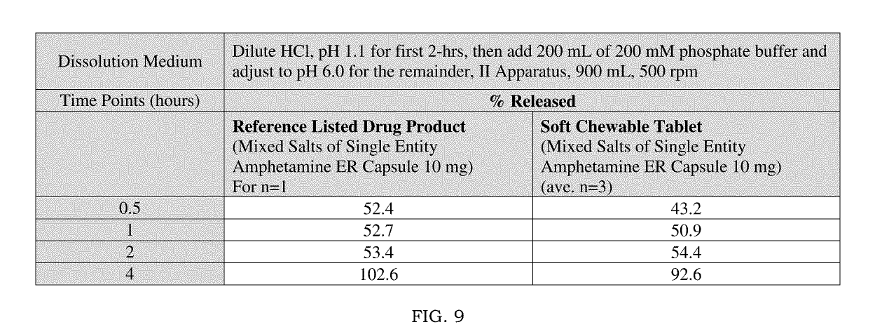

An active example was prepared using amphetamine from Adderall XR® capsules. The formulation is shown in Table 3. Physical properties are summarized in Table 4. FIG. 9 shows the results of comparative dissolution on a simulated gastric solution, of dilute HCl and phosphate buffer solution. The comparative dissolution implies the product of Example G may be bioequivalent to the capsules. Yet, the product of Example G has several abuse deterrent features. The softness of the tablets precludes pulverization for snorting or dosage concentration, and the presence of sodium lauryl sulfate (a surfactant), which is a nasal irritant. The blue color conjugated to the active agent and PEG are also abuse deterrent features.

TABLE 3
Quantitative Formulation Example G

| Ingredient | Example G (% w/w) |
|---|---|
| Amphetamine Mixed Salt (Contents equivalent | 16.2 |
| to 10 mg of Adderall XR Contents) | |
| Intragranular Components | |
| Preg. Maize Starch (Starch 1500) | 31.1 |
| Flavor (Artificial Chocolate) | 1.6 |
| Flavor (Artificial Vanilla) | 1.7 |
| Flavor (Bitter Masking Flavor) | 1.2 |
| Compressible Sugar | 2.4 |
| Sodium Lauryl Sulfate | 0.2 |
| Croscarmellose Sodium NF | 3.9 |
| PEG 3350 | 2.4 |
| FD&C Blue 1 | 0.1 |
| Granulation Aid | |
| Glycerin | 15.7 |
| Soybean Oil | 5.5 |
| Zea Mays Oil & BHA & BHT | 0.1 |
| Extragranular Components | |
| Silicified Microcrystalline Cellulose | 6.2 |
| Maltodextrin | 6.3 |
| Modified Corn Starch | 1.6 |
| Poly(ethylene) Oxide Polyox WSR 301 LEO | 2.4 |
| Lubrication & Flow Enhancement | |
| Magnesium Stearate | 0.8 |
| Aerosil 200 | 0.8 |
| Total | 100 |

### Procedure for Example G

1. All intragranular ingredients were mixed together in a poly-bag and passed through a sifting screen, followed by mixing again in poly-bag for uniform mixing.

2. All extragranular ingredients were mixed together in a poly-bag and passed through a sifting screen, followed by mixing again in poly-bag for uniform mixing.

3. Zea Mays Oil & BHA & BHT was added to the soybean oil and mixed uniformly.

The glycerin was added and mixed uniformly.

4. The granulation aid components from step 3 were added to intragranular blend from step 1 and mixed uniformly to obtain a granulate. The granules from this step were passed through a sifting screen.

5. The sifted granules from step 4 were mixed with approximately half of the extragranular blend from step 2 and mixed thoroughly form uniform mixing, followed by passing through a fine sifting screen.

6. Final granules from step 5 were mixed with the remaining extragranular blend from step 3 and mixed thoroughly and passed through the sifting screen used in step 5.

7. Pellets of two capsules of dextroamphetamine saccharate, amphetamine aspartate, dextroamphetamine sulfate, amphetamine sulfate (mixed salts of single entity amphetamine) extended-release (XR) thirty milligram (30 mg) capsule, manufactured by Shire US Inc. (Lot# AF8034A, Exp: August 2020) were taken out of their capsule shell and mixed together and weighed. Total weight was (368+369)=737 mg.

8. About one-hundred-twenty-two milligrams (122 mg) of the pellets from step 7 were mixed with six-hundred-twenty milligrams (620 mg) of granules from step 6 in and mixed uniformly.

9. The material from step 8 was compressed into tablets using a 17.6 mm×7.5 mm modified capsule shaped punch. The tablet from this step did not register any hardness on the hardness tester. The tablets had the characteristics described in Table 4. The table shown in FIG. 9 shows comparative dissolution on a simulated gastric solution, of dilute HCl and phosphate buffer solution.

TABLE 4
Product Characterization - Example G

| Description | | Units | Example G |
|---|---|---|---|
| Weight (avg. for 5-units) | | mg | 725 |
| Hardness (avg. for 5-units) | | kp | Not Registered |
| Length × Breadth (avg, for 5-units) | | mm | 17.8 × 7.7 |
| Thickness (avg. for 5-units) | | mm | 6.4 |
| Friability | 100 revs. | % w/w | 0.02 |
| | 200 revs. | loss | 0.03 |
| | 300 revs. | | 0.04 |
| Disintegration Time | | Minutes | 16 |
| Chewy | | — | Yes |
| Gritty | | — | No |
| Pin Penetration (By hand, using 0.40 | | — | Yes |
| mm dia. pin) without breaking tablet | | | |
| Grinding Study | Particle size | % Retained | 56 (% |
| | after grinding | on 35 Mesh | w/w) |
| | for 30-seconds | (≥500 μm) | |
| | | % Collected | 44 (% |
| | | in Pan | w/w) |
| | | (<500 μm) | |

Although the above description presents features of preferred embodiments of the present invention, other preferred embodiments may also be created in keeping with the principles of the invention. Furthermore, these other preferred embodiments may in some instances be realized through a combination of features compatible for use together despite having been presented independently in the above description.

The preferred forms of the invention described above are to be used as illustration only, and should not be utilized in a limiting sense in interpreting the scope of the present invention. Obvious modifications to the exemplary embodiments, as hereinabove set forth, could be readily made by those skilled in the art without departing from the spirit of the present invention.

The inventors hereby state their intent to rely on the Doctrine of Equivalents to determine and assess the reasonably fair scope of the present invention as pertains to any apparatus not materially departing from but outside the literal scope of the invention as set forth in the following claims.

1. A solid, abuse-resistant, edible, semi-plastic unit dosage form comprising:
a compressed tablet,
said tablet including a pharmaceutically active drug substance with abuse-potential,
said tablet having a hardness of less than about two kiloponds (2 kp) when measured on tablet hardness tester;
said tablet having a friability of less than about one percent (1%) at one-hundred (100) rotations.

2. The dosage form of as claimed in claim 1,
said tablet including an ingredient that reduces the abuse-potential of the pharmaceutically active drug substance,
said drug substance and said ingredient being conjugated.

3. The dosage form of as claimed in claim 1,
said tablet including an ingredient that regulates the: chemical stability; solubility; bioavailability; palatability; or combinations thereof of the pharmaceutically active drug substance,
said pharmaceutically active drug substance and said ingredient being conjugated.

4. The dosage form as claimed in claim 1,
said tablet including at least one auxiliary substance selected from the group consisting of:
a. at least one substance which irritates the nasal passages and/or pharynx;
b. at least one viscosity-increasing agent, which, with the assistance of a necessary minimum quantity of an aqueous liquid forms a gel which remains visually distinguishable when introduced into a further quantity of an aqueous liquid;
c. at least one antagonist for the pharmaceutical active drug substance;
d. at least one emetic;
e. at least one dye as an aversive agent; and
f. at least one bitter substance.

5. The dosage form as claimed in claim 1,
said active drug substance having a functional coating,
said coating being an extended-release coating, a delayed-release coating, a controlled-release coating, a film-coating, a barrier coating, an abuse deterrent coating, or combinations thereof.

6. The dosage form as claimed in claim 1,
said tablet including at least one ingredient that is a cyclodextrin, surfactant, solubility modulator, bioavailability modulator, or combination thereof,
said active drug substance and said at least one ingredient being conjugated.

7. The dosage form as claimed in claim 1,
said pharmaceutical active drug substance being incorporated into a drug delivery system.

8.-24. (canceled)

25. A process for the manufacture of an abuse deterrent, edible, semi-plastic unit oral dosage form, manufactured without molding or extrusion, for the oral administration of abuse potential pharmaceutical active ingredient, having a hardness of less than two kiloponds (2 kp), when tested using a conventional tablet hardness tester, and having a friability of less than one percent (1%) at hundred (100) rotations, comprising the steps of:
a. mixing at least one abuse-potential active ingredient with a fluid premix or a dry ingredient mixture;
b. blending dry ingredients comprising a bulking agent, a lipid, a flavoring agent, a disintegrating agent, a binding agent, a surfactant, a preservative, a lubricating agent, and an anti-sticking agent, or a mixture thereof, to form a dry ingredient mixture;
c. blending the fluid premix and the dry ingredient mixture to form a soft-chew mass;
d. sifting the soft-chew mass through at least one sifting screen to form granules of the soft-chew mass;
e. adding a lubricant or anti sticking agent to the granules of the soft-chew mass;
f. compressing the granules of the soft-chew mass in a tablet press to from soft chewable tablets.

26. (canceled)

27. (canceled)

28. The process of claim 25, where the dry ingredient mixture or the granulated compacted soft-chew mass are sifted through sifting equipment using impaction, attrition, compression or cutting.

29. The process of claim 25, where the dry ingredient mixture or the granulated compacted soft-chew mass are uniformly mixed using equipment using diffusion mixing, convection mixing or pneumatic mixing.

30.-37. (canceled)

38. The process of claim 25, wherein at least one of said dry ingredients has more than one function.

39. The process of claim 25, wherein the fluid premix includes at least one liquid component weight per weight of about five percent (5%) to about fifty percent (50%) of the dosage form.

40. The process of claim 39, wherein said fluid premix has more than one function.

41. The process of claim 40, wherein the liquid component includes an oil.

42. The process of claim 40, wherein the liquid component includes a humectant.

43. The process of claim 40, wherein the liquid component includes an oil and a humectant.

44. The process of claim 25, further comprising the step of:
drying the granules of the soft-chew mass at controlled temperature of less than fifty degrees Celsius (50□ C).

45. The process of claim 44, wherein the granules of the soft-chew mass is dried by equipment where heat is applied directly to a static solid bed, directly to a moving solid bed, or directly to a fluidized solid bed.

46. The process of claim 44, wherein the granules of the soft-chew mass is dried by equipment using indirect conduction heat applied to a static solid bed, a moving solid bed, or a fluidized solid bed.

47. A solid, abuse-resistant, edible, semi-plastic unit oral dosage form comprising:
a compressed tablet,
said tablet including a pharmaceutically active drug substance with abuse potential;
said tablet including an abuse-deterrent ingredient,
said tablet form having a hardness of less than about two kiloponds (2 kp).

48. The dosage form as claimed in claim 47,
said abuse-deterrent ingredient being a substance that promotes the chemical stability, increases or decreases the solubility, modulates the bioavailability, or increases or decreases the palatability of the pharmaceutically active drug substance.

49. The dosage form as claimed in claim 47,
said abuse-deterrent ingredient comprising at least one substance from the group consisting of:
a. at least one substance which irritates the nasal passages;
b. at least one substance which irritates the pharynx;
c. at least one substance which irritates the nasal passages and the pharynx;
d. at least one viscosity-increasing agent;
e. at least one antagonist for pharmaceutical active drug substance;
f. at least one emetic;
g. at least one dye; and
h. at least one bitter substance.

50. The dosage form as claimed in claim 47,
said pharmaceutically active drug substance having a functional coating,
said coating being an extended-release coating, a delayed-release coating, a controlled-release coating, a film-coating, a barrier coating, an abuse deterrent coating, or a combination of the foregoing.

51. The dosage form as claimed in claim 47,
said tablet having a hardness of less than about one kilopond (1 kp).

52. The dosage form as claimed in claim 51,
said tablet having a hardness of about zero kilopond (0 kp).

53. The dosage form as claimed in claim 47,
said tablet having a friability of less than about one percent (1%) at about one-hundred (100) rotations.

54. The dosage form as claimed in claim 53,
said tablet having a friability of less than about one percent (1%) at about two-hundred (200) rotations.

55. The dosage form as claimed in claim 54,
said tablet having a friability of less than about one percent (1%) at about three-hundred (300) rotations.

56. The dosage form as claimed in claim 47,
said tablet yielding a particulate when ground for about two (2) minutes,
said particulate having at least fifty percent (50%) cumulative particles presenting a particle size of at least five-hundred micrometers (500 μm).

57. The dosage form as claimed in claim 47,
said tablet having a disintegration time of less than about sixty (60) minutes according to USP disintegration test <701> using water as medium.
